(19) United States
(12) Patent Application Publication
(10) Pub. No.: US 2021/0205182 A1
(43) Pub. Date: Jul. 8, 2021

(54) DENTAL MOLDED ARTICLE AND DENTAL RESIN MATERIAL

(71) Applicant: UNIX-JAPAN CO., LTD., Nagoya-shi, Aichi (JP)

(72) Inventor: Hiroshi ANDO, Konan-shi, Aichi (JP)

(21) Appl. No.: 17/041,245

(22) PCT Filed: Mar. 25, 2019
(86) PCT No.: PCT/JP2019/012391

§ 371 Date: Sep. 24, 2020

(30) Foreign Application Priority Data
Mar. 30, 2018 (JP) 2018-068156

(51) Int. Cl. A61K 6/891; A61C 13/01; A61C 13/087; A61C 13/08; A61K 6/78; A61K 6/76; A61K 6/60; A61K 6/889

(52) U.S. Cl. CPC A61K 6/891; A61C 13/04; A61C 13/087; A61K 6/889; A61K 6/78; A61K 6/76; A61K 6/60; A61C 13/082

(57) ABSTRACT

A dental molded article has a predetermined shape so as to be used in an oral cavity, and contains polycaprolactone, acrylic resin, powdery silica, ascorbic acid, titanium oxide, a pigment, and zinc stearate. The acrylic resin is set to have a weight ratio of 5 to 30%, the powdery silica is set to have a weight ratio of 5 to 25%, the ascorbic acid is set to have a weight ratio of 0.3 to 1.8%, the titanium oxide is set to have a weight ratio of 0.001 to 0.02%, the pigment is set to have a weight ratio of 0.001 to 0.1%, and the zinc stearate is set to have a weight ratio of 0.001 to 0.1%, with respect to the polycaprolactone. Thus, a dental molded article and a dental resin material that can easily fit a mucosal surface in an oral cavity of a patient, are provided.

### CROSS-REFERENCE TO RELATED APPLICATIONS

This present application is a National Stage entry of International Application No. PCT/JP2019/012391, filed Mar. 25, 2019, which claims priority to Japanese Patent Application No. 2018-068156 filed Mar. 30, 2018. These prior applications are incorporated by reference in their entireties.

### FIELD OF THE INVENTION

This invention relates to a dental molded article and a dental resin material.

### BACKGROUND OF THE INVENTION

As a dental molded article, a denture base of a denture (false tooth), a dental prosthesis, a dental temporary crown, an artificial tooth, and the like are known (for example, see Patent Document 1).

### CITATION LIST

### Patent Document

Patent Document 1: Japanese Patent No. 5055397

### SUMMARY OF INVENTION

### Problems to be Solved by the Invention

A denture base that typifies the dental molded article is usually produced through impression taking (molding) by a dentist. However, for example, non-uniform thickness and hardness of a movable mucosal area of a jaw has made it difficult to enhance accuracy of molding obtained by the impression taking. Thus, the produced denture base does not fit in an oral cavity of a patient in many cases, also depending on individual differences of each patient and a degree of proficiency of a dentist who is an operator. In a case where the produced denture base does not fit in an oral cavity of a patient, the denture base is not expected to be stably placed in the oral cavity of the patient, and may likely be detached during, for example, eating and drinking.

An object of this invention is to provide a dental molded article and a dental resin material which can easily fit a mucosal surface and gums in an oral cavity of a patient even if stable fitting into the oral cavity of the patient cannot be expected due to inaccurate impression taking.

### Solution to the Problems and Effects of the Invention

In order to solve such a problem, a dental molded article according to a first aspect of this invention is directed to a dental molded article that has a predetermined shape, that is used in an oral cavity, and that contains polycaprolactone. The dental molded article includes: acrylic resin made of a polymer of acrylic acid ester, a polymer of methacrylic acid ester, or a copolymer of acrylic acid ester and methacrylic acid ester, the acrylic resin having a weight ratio of 5 to 30% with respect to the polycaprolactone; powdery silica having a weight ratio of 5 to 25% with respect to the polycaprolactone; ascorbic acid having a weight ratio of 0.3 to 1.8% with respect to the polycaprolactone; titanium oxide having a weight ratio of 0.001 to 0.02% with respect to the polycaprolactone; a pigment having a weight ratio of 0.001 to 0.1% with respect to the polycaprolactone; zinc stearate having a weight ratio of 0.001 to 0.1% with respect to the polycaprolactone; and a remainder being the polycaprolactone and unavoidable impurities.

A dental molded article according to a second aspect of this invention is directed to a dental molded article that has a predetermined shape, that is used in an oral cavity, and that contains polycaprolactone. The dental molded article includes: thermoplastic polyester resin having a weight ratio of 5 to 30% with respect to the polycaprolactone; powdery silica having a weight ratio of 5 to 25% with respect to the polycaprolactone; ascorbic acid having a weight ratio of 0.3 to 1.8% with respect to the polycaprolactone; titanium oxide having a weight ratio of 0.001 to 0.02% with respect to the polycaprolactone; a pigment having a weight ratio of 0.001 to 0.1% with respect to the polycaprolactone; zinc stearate having a weight ratio of 0.001 to 0.1% with respect to the polycaprolactone; and a remainder being the polycaprolactone and unavoidable impurities.

More preferably, a dental molded article according to the first aspect of this invention described above is directed to a dental molded article that has a predetermined shape, that is used in an oral cavity, and that contains polycaprolactone. The dental molded article includes: acrylic resin having a weight ratio of 10 to 25% with respect to the polycaprolactone; powdery silica having a weight ratio of 5 to 20% with respect to the polycaprolactone; ascorbic acid having a weight ratio of 0.3 to 1.8% with respect to the polycaprolactone; titanium oxide having a weight ratio of 0.001 to 0.02% with respect to the polycaprolactone; a pigment having a weight ratio of 0.001 to 0.1% with respect to the polycaprolactone; zinc stearate having a weight ratio of 0.001 to 0.1% with respect to the polycaprolactone; and a remainder being the polycaprolactone and unavoidable impurities.

More preferably, a dental molded article according to the second aspect of this invention described above is directed to a dental molded article that has a predetermined shape, that is used in an oral cavity, and that contains polycaprolactone. The dental molded article includes: thermoplastic polyester resin having a weight ratio of 10 to 25% with respect to the polycaprolactone; powdery silica having a weight ratio of 5 to 20% with respect to the polycaprolactone; ascorbic acid having a weight ratio of 0.3 to 1.8% with respect to the polycaprolactone; titanium oxide having a weight ratio of 0.001 to 0.02% with respect to the polycaprolactone; a pigment having a weight ratio of 0.001 to 0.1% with respect to the polycaprolactone; zinc stearate having a weight ratio of 0.001 to 0.1% with respect to the polycaprolactone; and a remainder being the polycaprolactone and unavoidable impurities.

Furthermore, in order to solve the aforementioned problem, a dental resin material according to a first aspect of this invention is directed to a dental resin material that is used in accordance with a dental molded article used in an oral cavity, and that contains polycaprolactone. The dental resin material includes: acrylic resin made of a polymer of acrylic acid ester, a polymer of methacrylic acid ester, or a copolymer of acrylic acid ester and methacrylic acid ester, the acrylic resin having a weight ratio of 5 to 30% with respect to the polycaprolactone; powdery silica having a weight ratio of 5 to 25% with respect to the polycaprolactone; and a remainder being the polycaprolactone and unavoidable impurities.

Furthermore, in order to solve the aforementioned problem, a dental resin material according to a second aspect of this invention is directed to a dental resin material that is used in accordance with a dental molded article used in an oral cavity, and that contains polycaprolactone. The dental resin material includes: thermoplastic polyester resin having a weight ratio of 5 to 30% with respect to the polycaprolactone; powdery silica having a weight ratio of 5 to 25% with respect to the polycaprolactone; and a remainder being the polycaprolactone and unavoidable impurities.

More preferably, a dental resin material according to the first aspect of this invention described above is directed to a dental resin material that is used in accordance with a dental molded article used in an oral cavity, and that contains polycaprolactone. The dental resin material includes: acrylic resin having a weight ratio of 10 to 25% with respect to the polycaprolactone; powdery silica having a weight ratio of 5 to 20% with respect to the polycaprolactone; and a remainder being the polycaprolactone and unavoidable impurities.

More preferably, a dental resin material according to the second aspect of this invention described above is directed to a dental resin material that is used in accordance with a dental molded article used in an oral cavity, and that contains polycaprolactone. The dental resin material includes: thermoplastic polyester resin having a weight ratio of 10 to 25% with respect to the polycaprolactone; powdery silica having a weight ratio of 5 to 20% with respect to the polycaprolactone; and a remainder being the polycaprolactone and unavoidable impurities.

The dental molded article and the dental resin material (hereinafter, referred to as the dental molded article and the like when collectively called) according to this invention contain polycaprolactone, acrylic resin (or thermoplastic polyester resin instead of the acrylic resin) made of a polymer of acrylic acid ester, a polymer of methacrylic acid ester, or a copolymer of acrylic acid ester and methacrylic acid ester, and powdery silica, as the resin component. The acrylic resin (or thermoplastic polyester resin) has a weight ratio of 5 to 30% with respect to the polycaprolactone, the powdery silica has a weight ratio of 5 to 25% with respect to the polycaprolactone, and the remaining resin component is polycaprolactone.

The component itself of the acrylic resin is well known. Examples of the polymer (methacrylic resin) of methacrylic acid ester include a polymer of methyl methacrylate (abbreviated as MMA) and a polymer of ethyl methacrylate (abbreviated as EMA). Examples of the polymer of acrylic acid ester include a polymer of methyl acrylate (abbreviated as MA) and a polymer of ethyl acrylate (abbreviated as EA). A copolymer of acrylic acid ester and methacrylic acid ester is obtained by copolymerization of both of the esters.

Thermoplastic polyester resin itself is also well-known. For example, a commercially available thermoplastic polyester such as polyethylene terephthalate (PET resin), polybutylene terephthalate (PBT resin), and polyethylene naphthalate (PEN resin) can be used.

The powdery silica (filler) exerts an influence on the viscosity of a material of the dental molded article and the like, and on, for example, the hardness and the bending strength in conjunction with the contained polycaprolactone. The powdery silica has an indefinite shape, a spherical shape, or a cotton-like shape, and has, for example, particle sizes of about 1 to 100 nm.

Therefore, the dental molded article and the like according to this invention have a melting point of about 60 to 85° C. while having a high bending strength and a high tensile strength at normal temperatures. Therefore, in a case where, for example, a dentist places the denture base as the dental molded article according to this invention in an oral cavity of a patient, the dentist immerses the denture base in hot water having a temperature of about 70 to 75° C. for about 30 seconds to soften the denture base, and strongly presses the softened denture base onto a mucosal surface in the oral cavity of the patient, whereby the inner surface (back face) of the denture base can fit the shape of the mucosal surface in the oral cavity (fit an alveolar ridge). Therefore, a drawback in impression taking can be compensated for. Meanwhile, a denture base that has high adhesion and can fit a mucosal surface in an oral cavity of a patient can be easily produced without taking an impression from the patient.

Furthermore, a patient immerses the dental resin material in hot water having a temperature of about 70 to 75° C. for about 30 seconds to soften the dental resin material, adheres the softened dental resin material to an inner surface (back face) of an existing denture, and strongly presses the dental resin material onto a mucosal surface in an oral cavity, by her/himself, whereby removal of the denture from the oral cavity can be advantageously prevented due to the adhering effect. That is, the dental resin material according to this invention can substitute for a so-called false tooth stabilizer.

As described above, the dental molded article and the like according to this invention contains at least about 45% of polycaprolactone. The polycaprolactone is a biodegradable resin material, and has a partially crystalline form (semi-crystalline polymer) and can chemically bond firmly to another amorphous resin. However, in a case where a molded article which is solely made of the homopolymer of polycaprolactone is used in an oral cavity, significant hydrolysis is caused by oxidation, and the molded article can be used only for a short period. Meanwhile, polycaprolactone has a low melting point (about 60° C.), and allows the shape of the molded article to be freely changed by immersing the molded article in hot water having a temperature of not lower than the melting point. According to this invention, the dental molded article and the like that can be used in an oral cavity for a long period have been found by utilizing the properties of polycaprolactone and overcoming the disadvantages of polycaprolactone.

In the dental molded article and the like according to this invention, the acrylic resin (or the thermoplastic polyester resin instead of the acrylic resin) has a weight ratio of 5 to 30% with respect to the polycaprolactone, and the powdery silica has a weight ratio of 5 to 25% with respect to the polycaprolactone. The acrylic resin (or the thermoplastic polyester resin) preferably has a weight ratio of 10 to 25% with respect to the polycaprolactone and the powdery silica preferably has a weight ratio of 5 to 20% with respect to the polycaprolactone. Effectively, a weight ratio of both the acrylic resin (or the thermoplastic polyester resin) and the powdery silica with respect to the polycaprolactone is 20 to 40%, and, more effectively, the weight ratio thereof is 30 to 35%.

In a case where the acrylic resin (or the thermoplastic polyester resin) has a weight ratio of 5 to 30% with respect to the polycaprolactone, and a remaining resin component is polycaprolactone, while the resin can be prevented from deteriorating due to hydrolysis of polycaprolactone in an oral cavity, and the dental molded article and the like can have a hardness proper for a human body, occurrence of separation and breakage such as removal and fracture can be prevented when the dental molded article and the like are placed in the oral cavity, and, furthermore, increase of the melting point can be inhibited. The dental molded article and the like according to this invention have a melting point of about 65 to 80° C. These effects are exerted in a preferable manner when the acrylic resin (or the thermoplastic polyester resin) has a weight ratio of 5 to 30% with respect to the polycaprolactone, and the powdery silica has a weight ratio of 5 to 25% with respect to the polycaprolactone, and are exerted in a more preferable manner when the acrylic resin (or the thermoplastic polyester resin) has a weight ratio of 10 to 25% with respect thereto, and the powdery silica has a weight ratio of 5 to 20% with respect thereto.

In a case where the acrylic resin (or the thermoplastic polyester resin) has a weight ratio of less than 5% with respect to the polycaprolactone, the resin is likely to deteriorate due to hydrolysis of the polycaprolactone. Meanwhile, in a case where the acrylic resin (or the thermoplastic polyester resin) has a weight ratio of greater than 30% with respect to the polycaprolactone, the denture base body made of amorphous resin has a lower chemical bonding force, and is highly likely to be separated or broken. Such characteristics are improper for the dental molded article. Furthermore, a melting point increases, to reduce workability for changing a shape.

Ascorbic acid that is one of components added in the dental molded article and the like according to this invention prevents oxidation of the dental molded article and the like, thereby improving durability, so that the dental molded article and the like can be allowed to suit the environment in an oral cavity and maintain their function for a long period. In a case where a weight ratio with respect to the polycaprolactone is less than 0.3%, oxidation cannot be sufficiently prevented. Meanwhile, in a case where the weight ratio is greater than 1.8%, the effect is saturated. Thus, a weight ratio of the ascorbic acid with respect to the polycaprolactone was set to be 0.3 to 1.8%. The weight ratio is preferably 0.6 to 1.2%.

In a case where the color of the dental molded article is made close to a color of gums or the like in an oral cavity, the resin is dyed pink. Titanium oxide and the pigment (for example, red pigment) are added for coloring (color development). For example, a mixing ratio between the red pigment and titanium oxide (white color) is adjusted in order to generate a color (pink) closer to actual gums.

The content of each of titanium oxide and the pigment is determined according to a color with which the dental molded article is mainly dyed. Titanium oxide has a weight ratio of 0.001 to 0.02% with respect to the polycaprolactone. In a case where the weight ratio is less than 0.001%, titanium oxide cannot function to adjust the color with respect to the pigment. Meanwhile, in a case where the weight ratio is greater than 0.02%, the titanium oxide degrades physical properties such as a strength of the resin material so as to deviate from color adjusting function. Therefore, in a case where the color of the dental molded article is made close to a color of gums or the like in an oral cavity, the weight ratio of the titanium oxide with respect to the polycaprolactone is set to be 0.001 to 0.02%. The weight ratio is preferably 0.003 to 0.01%.

The pigment (for example, red) has a weight ratio of 0.001 to 0.1% with respect to the polycaprolactone. In a case where the weight ratio is less than 0.001%, an effect of dyeing with a color close to a color of gums is not obtained. Meanwhile, in a case where the weight ratio is greater than 0.1%, the target dyeability is reduced and the weight ratio is not preferable for the physical properties of the resin material. Therefore, in a case where the color of the dental molded article is made close to a color of gums or the like in an oral cavity, the weight ratio of the pigment with respect to the polycaprolactone is set to be 0.001 to 0.1%. The weight ratio is preferably 0.001 to 0.008%.

The zinc stearate contributes to uniform mixing and stirring of the titanium oxide, the pigment, and the polycaprolactone resin, that is, contributes to uniform dyeability for uniform coloring, to enhance stirring and mixing of the additives with each other. The zinc stearate has a weight ratio of 0.001 to 0.1% with respect to the polycaprolactone in a case where the pigment (for example, red pigment) is added. In a case where the weight ratio is less than 0.001%, an effect of enhancing stirring and mixing during dyeing cannot be expected. Meanwhile, in a case where the weight ratio is greater than 0.1%, stirring and mixing themselves become difficult. Therefore, in a case where the color of the dental molded article is made close to a color of gums or the like in an oral cavity, the weight ratio of the zinc stearate with respect to the polycaprolactone is set to be 0.001 to 0.1%. The weight ratio is preferably 0.001 to 0.05%. Also in a case where, for example, magnesium stearate is used in addition to or instead of the zinc stearate, the same effect can be obtained.

Thus, by adding zinc stearate, titanium oxide, and the pigment, dyeing proper for the dental molded article can be performed, and, furthermore, fluidity of resin during injection molding increases, thereby improving reproducibility of the dental molded article in detail. In addition, in a case where the dental molded article is attached/detached in an oral cavity, the above-described additives allow reduction of resistance caused by friction between residual tissues and teeth in the oral cavity of the patient, and the dental molded article, and good lubrication is obtained. As a result, the dental molded article can be easily attached and detached in an oral cavity of a patient.

The dental molded article may be formed in any one selected from, for example, a denture base, a dental prosthesis, a dental temporary crown, and an orthodontic appliance.

The denture base is, for example, a denture base of full dentures or a denture base of a partial denture, and supports artificial teeth and functions as a placement portion in an oral cavity. More specifically, examples of the denture base include a denture base with a plate (non-clasp denture base) in which a wire (retainer in an oral cavity: also called clasp) and metal are not used. Such a denture base is usually colored pink so as to correspond to a color of gums, and a red pigment is used for the coloring, and titanium oxide is used as an additive, having a white color, to be mixed with the red pigment. Iron oxide as an additive having a dim color may be added to the red pigment, in order to generate a natural color that is close to a color of actual gums.

The dental prosthesis is a very thin prosthesis (aesthetic laminate) that is adhered to an anterior tooth of a patient to enhance an aesthetic effect, or the dental prosthesis is a restorative material used for restoring a significantly broken tooth. Examples of the dental prosthesis include an inlay for restoring the entirety or a part of teeth, an artificial tooth crown (veneer crown), and a bridge that is placed as a cover for bridging with the use of abutment teeth obtained by grinding teeth adjacent to an extracted tooth. The crown restorative material is a material for restoring an artificial tooth crown or a natural tooth crown which has been broken and damaged just in an oral cavity, and the product of this invention can be utilized as the crown restorative material.

The dental prosthesis, the dental temporary crown, and the orthodontic appliance need not necessarily be colored red so as to correspond to a color of gums. However, in order to, for example, prevent the existence thereof from being conspicuous, coloring can be performed so as to correspond to a color of gums, similarly to the denture base. In a case where the dental temporary crown and the orthodontic appliance are not colored so as to correspond to a color of gums, a pigment for a desired color may be used together with titanium oxide or iron oxide to perform coloring with any color as appropriate.

The dental temporary crown is, for example, a mouthpiece, a temporary tooth that is provisionally placed after extraction of a tooth, and a veneer crown that is placed as a cover until an implant is stabilized after the implant surgery, and they are not placed for a long period but are used in a certain limited period (temporarily). The orthodontic appliance is placed in an oral cavity for, for example, orthodontic treatment for occlusion between an upper jaw and a lower jaw, for a dental plate, and for teeth alignment and teeth angles.

In addition, the dental molded article of this invention can be used as a substitute for a retainer (clasp) for retaining a partial denture (partial false tooth), a unit (palatal bar) that acts as a bridge between back teeth of an upper jaw, or the like.

A metal allergy concern is overcome in a case where the dental molded article such as the above-described denture base, dental prosthesis, dental temporary crown, and orthodontic appliance is made of resin that contains polycaprolactone as a main component. Furthermore, the water absorption is extremely low, and, therefore, the dental molded article is unlikely to contain water and bacteria are less likely to remain, thereby maintaining hygiene. Meanwhile, in, for example, a non-clasp denture (denture base in which a wire and metal are not used as a retaining member) made of polyamide or nylon, adhesion between an artificial tooth and a denture base is not sufficient, so that food residue enters a boundary between the artificial tooth and the denture base and remains therebetween, to cause proliferation of bacteria. Therefore, hygiene is unlikely to be maintained. Meanwhile, the dental molded article according to this invention allows the artificial tooth and the denture base to chemically bond to each other with the use of a solvent, thereby effectively reducing proliferation of bacteria.

The dental molded article according to this invention may have a coating layer that is made of acrylic resin or epoxy resin and is located so as to cover the surface of the dental molded article in a state where the coating layer is adhered to the surface of the dental molded article. Thus, the surface of the dental molded article can be covered with the acrylic resin or the epoxy resin, and the surface of the dental molded article can be inhibited from becoming non-smooth in an oral cavity of a patient. Furthermore, since the coating layer is made of acrylic resin or epoxy resin, the coating layer can be adhered to polycaprolactone. Specifically, an existing acrylic or epoxy-based dental surface lubricant can be used to inhibit the surface of the dental molded article from becoming non-smooth.

The dental molded article according to this invention has excellent dimension stability. Therefore, a dentist is allowed to precisely adapt the dental molded article to a tooth model (impression) taken by the dentist. Particularly, in the case of a non-clasp denture, excellent wearability can be provided to a patient. The dental molded article of this invention has a high elastic modulus and has excellent stability as a denture base.

The dental molded article according to this invention as described above is produced by melting resin materials in various forms such as a pellet-like form, a sheet-like form, and a block-like form, and performing, for example, injection molding or packing molding. The dental molded article according to this invention is produced by melting or mixing polycaprolactone and acrylic resin (or thermoplastic polyester resin). Thus, resin allergy is not caused in a human body unlike in a case where only acrylic resin that causes resin allergy in a human body is a material. Similarly, unlike in a case where, for example, polycarbonate resin that causes elution of bisphenol A or the like is a material, the dental molded article that does not cause elution of bisphenol A or the like and that is excellent in safety can be produced. The dental molded article according to this invention can assure sufficient adhesion to another resin and is suitable for an environment in an oral cavity of a patient.

The dental resin material according to this invention can be used as, for example, a denture base liner, a denture base repairing material, a denture base reinforcing material, a dental prosthesis, and a crown restorative material. The denture base liner functions to, for example, re-adjust the height of the denture base. The denture base repairing material functions to restore a damaged portion of the denture base. The denture base reinforcing material functions to reinforce a portion, of the denture base, having an insufficient strength. The denture base liner is disposed on the inner surface side (the oral cavity mucosal surface side, the gum side) of the denture base, and, thus, color is not particularly limited. In a case where the denture base repairing material and the denture base reinforcing material are disposed on the inner surface side of the denture base, the colors are not particularly limited as in the denture base liner. However, in a case where the denture base repairing material and the denture base reinforcing material are disposed on the outer surface side of the denture base, coloring can be performed so as to correspond to a color of an oral cavity mucosa or gums.

In a case where the dental molded article according to this invention is used as a denture base, or in a case where an inner surface liner is formed, by a denture base liner as the dental resin material according to this invention, on a denture base body made of amorphous resin such as PMMA resin (polymethyl methacrylate), PET resin (polyethylene terephthalate), or PSU resin (polysulfone), since the denture base liner has a softening temperature of about 70 to 75° C., a patient is allowed to immerse the denture base in hot water having a temperature of about 70 to 75° C. to soften the inner surface of the denture base, and to strongly press the softened inner surface of the denture base onto the oral cavity, by him/herself, thereby placing the denture base body in the oral cavity.

In a case where the inner surface liner is formed, by the denture base liner as the dental resin material according to this invention, on a denture base body that is made of amorphous resin formed of PMMA only, for example, a dichloromethane primer or acrylic monomer is applied to the joining portion, thereby obtaining firm chemical bonding. The denture base liner as the dental resin material according to this invention has strong joining characteristics, whereby the amount thereof can be increased by softening.

In a case where the denture base reinforcing material as the dental resin material according to this invention is used for a conventional denture base made of PMMA only, the drawback of the conventional denture base that the denture base is easily broken, can be advantageously overcome. Furthermore, in a case where a non-clasp denture (denture base in which a wire and metal are not used as a retaining member) is produced as the dental molded article according to this invention, the produced molded article can be constructed and repaired. For example, in a case where a patient has ridge resorption (change in shape of edentulous jaw) after production of the non-clasp denture, a dentist changes the shape of the denture base through softening by her/himself, or uses the denture base liner as the dental resin material according to this invention to construct and repair the shape or the like of the inner surface of the denture base, thereby easily enhancing fitting accuracy.

In a case where a denture base used by a patient is not the dental molded article according to this invention, or in a case where a denture base body that is made of PMMA resin or the like and used by a patient does not have the inner surface liner formed by a denture base liner as the dental resin material according to this invention, the patient stabilizes the denture base body in the oral cavity by her/himself by using an existing false tooth stabilizer in general.

In this invention, the patient immerses a denture base stabilizer as the dental resin material according to this invention in hot water having a temperature of about 70 to 75° C. by her/himself to soften the denture base stabilizer, adheres the softened denture base stabilizer to the inner surface (back face) of the denture base used by the patient, and strongly presses the denture base stabilizer onto the mucosal surface in the oral cavity, thereby placing the denture base body in the oral cavity. The denture base stabilizer as the dental resin material according to this invention provides excellent wearability and assures sufficient stability for the denture base.

### BRIEF DESCRIPTION OF THE DRAWINGS

FIG. 1 illustrates a partial denture as a dental molded article according to this invention.

FIG. 2 illustrates an example in which the partial denture shown in FIG. 1 is placed in a human oral cavity.

FIG. 3 illustrates a crown representing an example of a dental prosthesis as the dental molded article according to this invention.

FIG. 4 illustrates an aesthetic laminate representing an example of a dental prosthesis as the dental molded article according to this invention.

FIG. 5 illustrates an example in which the aesthetic laminate shown in FIG. 4 is placed over a human anterior tooth.

FIG. 6 illustrates an example of an orthodontic appliance as the dental molded article according to this invention.

FIG. 7 illustrates an example in which the orthodontic appliance shown in FIG. 6 is placed in a human oral cavity.

FIG. 8 illustrates a mouthpiece representing an example of a dental temporary crown as the dental molded article according to this invention.

FIG. 9 illustrates an example in which a denture base repairing material as a dental resin material according to this invention is used.

FIG. 10 illustrates an example in which a denture base stabilizer as the dental resin material according to this invention is used.

FIG. 11 illustrates an example in which a denture base stabilizer as the dental resin material according to this invention is used.

### DESCRIPTION OF EMBODIMENTS

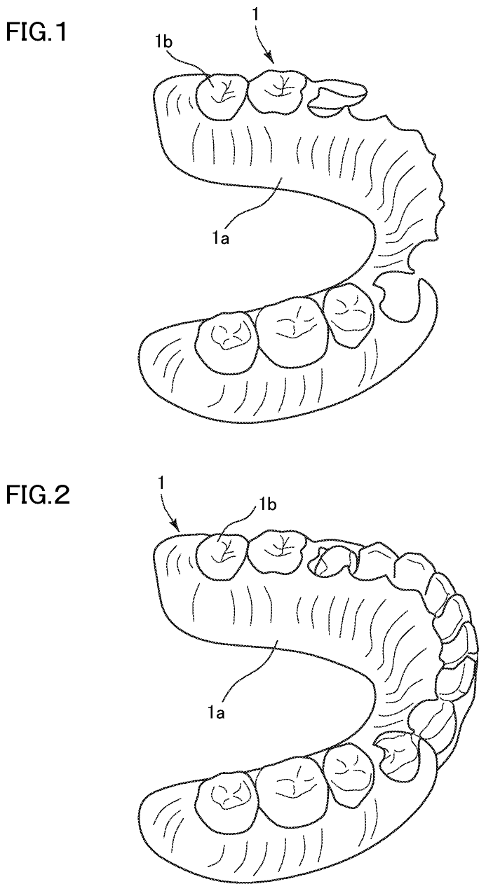

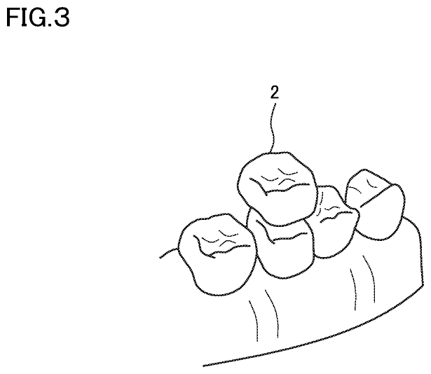

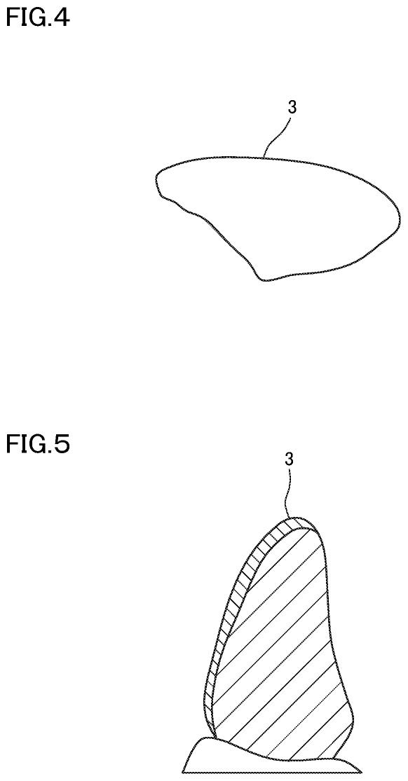

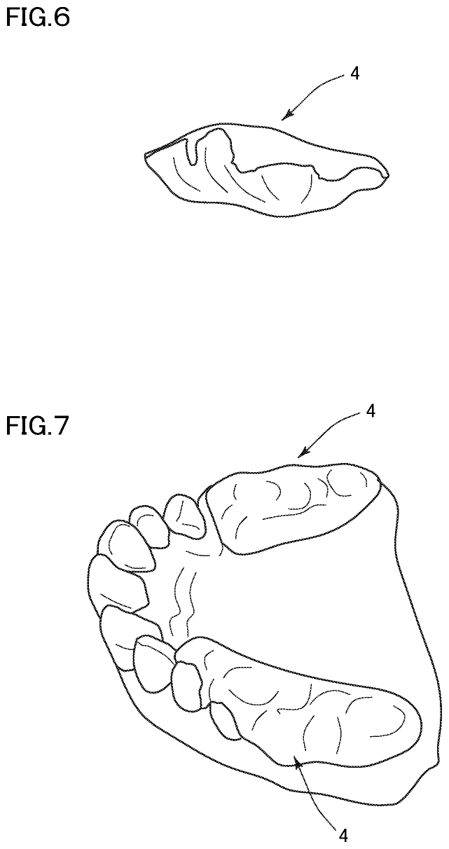

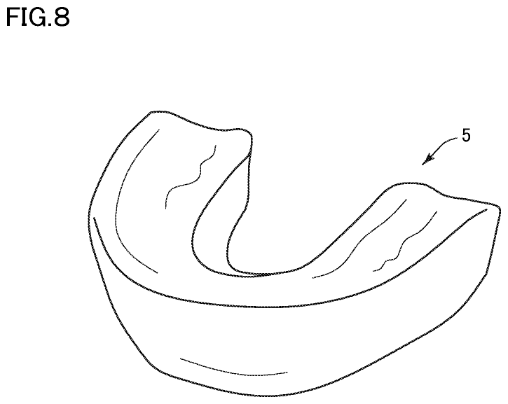

FIG. 1 to FIG. 8 each illustrate an example of a dental molded article according to this invention. FIG. 1 illustrates an example of a denture base 1a of a partial denture 1 (reference character 1b represents an artificial tooth). FIG. 2 illustrates an example in which the partial denture 1 is placed in a human oral cavity. FIG. 3 illustrates an example of a crown 2 (dental prosthesis). FIG. 4 illustrates an example of an aesthetic laminate 3 (dental prosthesis). FIG. 5 illustrates an example in which the aesthetic laminate 3 is placed over a human anterior tooth. FIG. 6 illustrates an example of an orthodontic appliance 4. FIG. 7 illustrates an example in which the orthodontic appliance 4 is placed in a human oral cavity. FIG. 8 illustrates an example of a mouthpiece (dental temporary crown).

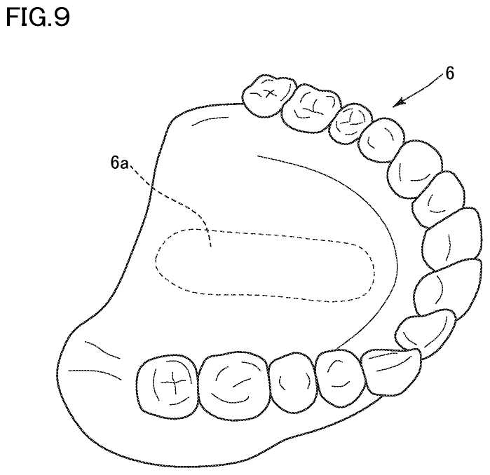

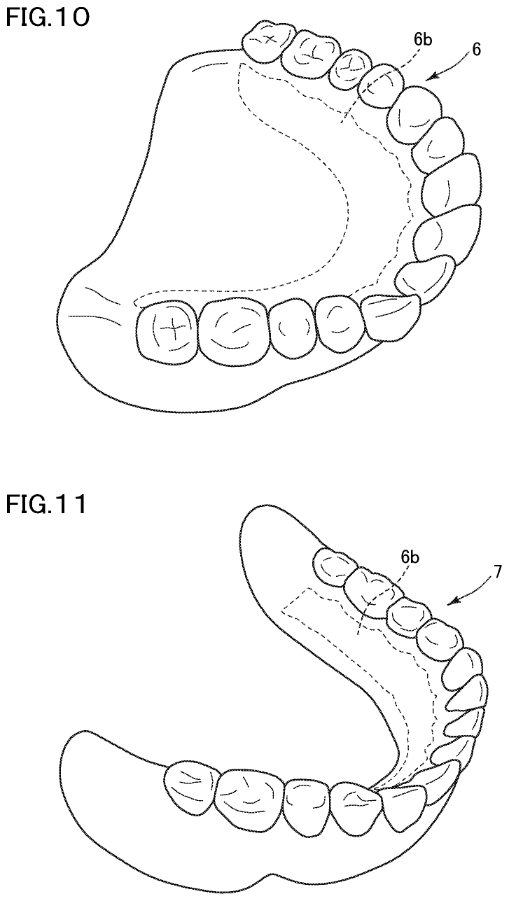

FIG. 9 to FIG. 11 each illustrate an example of a dental resin material according to this invention. FIG. 9 illustrates an example in which a denture base repairing material 6a is used (reference character 6 represents full dentures). FIG. 10 illustrates an example of the full dentures 6 for which a denture base stabilizer 6b is placed. FIG. 11 also illustrates an example of full dentures 7 for which the denture base stabilizer 6b is placed.

Next, examples of the dental resin material for producing such various kinds of the dental molded articles will be described.

### Example 1

Firstly, an unpolymerized acrylic monomer, for example, MMA was polymerized with a polymer of methacrylic acid ester, for example, PMMA which had a weight ratio of 70%, to obtain acrylic resin (containing unavoidable impurities). Instead of the methacrylic acid ester, acrylic acid ester may be used. Alternatively, a copolymer of acrylic acid ester and methacrylic acid ester may be used.

Next, polycaprolactone (PCL) obtained by synthesis of ε-caprolactone ([C6H10O2] (“2-oxepanone” homopolymer), and the acrylic resin having a weight ratio of 15% with respect to the polycaprolactone (PCL) were further copolymerized with each other to generate copolymerized polycaprolactone. Adjustment was performed in the obtained copolymerized polycaprolactone such that the weight ratio of ascorbic acid was 0.8%, the weight ratio of titanium oxide was 0.009%, the weight ratio of a red pigment (for example, one or more selected from organic and inorganic pigments such as aluminum lake and red iron oxide, which are certified colorants) was 0.003%, and the weight ratio of zinc stearate was 0.018%, with respect to the polycaprolactone (PCL).

Specifically, for use in a denture base gum part, the weight ratio of the acrylic resin is 20% (10 kg), the weight ratio of powdery silica is 15% (7.5 kg), the weight ratio of ascorbic acid is 0.8% (400 g), the weight ratio of titanium oxide is 0.009% (4.5 g), the weight ratio of the red pigment is 0.003% (1.5 g), and the weight ratio of zinc stearate is 0.018% (9 g), with respect to polycaprolactone (PCL) (50 kg).

The above-described materials (copolymerized polycaprolactone, ascorbic acid, titanium oxide, the red pigment, and zinc stearate) were blended by an extruder, and a colored pellet-like dental resin material (dental resin material used for producing the dental molded article substituting for gums and the like) was obtained. The dental resin material contains a very small amount of unavoidable impurities as a weight ratio with respect to the polycaprolactone. They have no relation with the intrinsic requirements and effects of this invention, and do not exert an influence thereon.

The dental molded articles such as the denture base 1a, the crown 2 (dental prosthesis), the aesthetic laminate 3 (dental prosthesis), the orthodontic appliance 4, and the mouthpiece 5 (dental temporary crown) as shown in FIG. 1 to FIG. 8 are obtained through molding from the generated dental resin material. In a case where such a dental molded article is molded, processing can be easily performed by using a typical general-purpose molding machine through any of injection molding, compression molding, vacuum pressure molding, and the like. The denture base 1a and the artificial tooth 1b shown in FIG. 1 are chemically bonded to each other by using a solvent, whereby food residue can be inhibited from entering a boundary between the denture base 1a and the artificial tooth 1b and remaining therebetween, thereby suppressing proliferation of bacteria.

In a case where the dental molded article according to this invention is produced by injection molding, for example, the dental resin material in pellet form is put into a barrel of a general-purpose resin molding machine, and the furnace temperature is set to 70 to 100° C. to soften and melt the raw material, thereby performing the injection molding. The dental resin material has zinc stearate blended therein and thus has high resin fluidity, and can allow the molded article to precisely exhibit reproducibility in detail.

The above-described dental resin material can be used as the denture base repairing material 6a, and the denture base stabilizers 6b, 6b as shown in FIG. 9 to FIG. 11, respectively. In this case, the dental resin material need not be formed into a specific shape, and may be formed into, for example, a quadrangular-plate-like shape, softened when used, and attached to the full dentures 6, 7.

The dental molded article and the dental resin material as described above have the following substantially advantageous characteristics and features.

1. Resin allergy reaction is not caused in human bodies.

2. Bisphenol A as a material that causes an endocrine disrupting action is not eluted, so that the dental molded article and the like can be used for human bodies with high safety.

3. The resin components are not eluted in an oral cavity with respect to all the additives, and, therefore, high safety can be provided and there is no safety concern about the additives and the pigments.

4. A polyester fiber material that is colored so as to simulate blood vessels in human oral cavities may be embedded in the molded article, for aesthetically increasing the naturalness of the denture base.

5. By blending zinc stearate as an additive, the pigments are completely and uniformly mixed during dyeing of resin, to substantially enhance dyeability.

6. By blending zinc stearate, lubrication of the surface of the resin itself increases, thereby increasing smoothness. This is effective for attaching and detaching the denture base body.

7. By blending titanium oxide as an additive, a natural hue can be reproduced so as to match human gums during dyeing of the denture base.

8. The dental molded article according to this invention has a mechanical strength, a bending strength, and heat resistance that are more excellent than or equivalent to those of a dental molded resin material (polycarbonate, polyamide, nylon, acrylic, or the like) that has been conventionally used, so that the denture base can be produced such that the denture base is unlikely to be broken, is very compact, and does not provide an uncomfortable feeling in an oral cavity. Furthermore, water absorption is very low as compared with a conventional resin material, and proliferation of bacteria and deterioration of the physical properties of resin due to water absorption are substantially reduced.

9. The dental molded article and the like according to this invention are unlikely to absorb water in air and can be stored at normal temperatures without problem. The dental molded article and the like need not be dried by heat for removing a water content during injection molding, and work efficiency is also excellent.

10. The dental molded article and the like according to this invention can be softened at a temperature of about 70 to 75° C. to freely change the shape, and can easily fit the form and structure of a mucosal surface, a tooth cervix, and a molded tooth.

The evaluation test was performed for the above-described dental molded article (dental molded article substituting for gums and the like) according to this invention, and the results are indicated in Table 1.

TABLE 1

| | Example 1 | Example 2 | polypropylene | polyamide | polycarbonate |
|---|---|---|---|---|---|
| molding temperature ° C. | 80~120 | 80~120 | 180~220 | 230~270 | 270~300 |
| molding shrinkage rate | 0.1~0.2 | 0.08~0.15 | 0.3~0.4 | 0.2~0.3 | 0.1~0.3 |
| attachability | good | good | slightly good | slightly good | slightly good |
| grinding feeling | good | good | poor | poor | slightly good |
| removability from plaster | good | good | good | good | slightly good |
| model | | | | | |
| flexural modulus Mpa | 3600 | 3800 | 2150 | 3500 | 2500 |
| bending before water | whitened when | whitened when | whitened when | broken when | broken when |
| absorption | bending was | bending was | bending was | bending was | bending was |
| | performed 250 | performed 250 | performed 200 | performed 120 | performed 5 |
| | or more times | or more times | times | times | times |
| bending after water | as above | as above | as above | as above | as above |
| absorption | | | | | |
| bending strength Mpa | 150 | 170 | 60 | 65 | 91 |
| elongation mm | not less than 15 | not less than 16 | not less than 12 | not less than 12 | not less than 12 |
| hardness Hv | 13 | 13 | 9.8 | 9.4 | 13.3 |
| water absorption rate wt % | 0.05~0.2 | 0.1~0.4 | 0.04~0.08 | 0.3~0.8 | 0.2~0.8 |
| coloring Δ E curry | 1.29 | 1.2 | 1.25 | 1.5 | 1.5 |
| coloring Δ E fuchsin | 5 | 5 | 5 | 9 | 9 |

As indicated in Table 1, for example, as to a result of measurement in the repeated-bending tests before and after water absorption, the dental resin material of example 1 was more excellent than other dental resin materials (polyamide and polycarbonate except for polypropylene). For example, in the repeated-bending test before water absorption, although the material made of polycarbonate was broken when the bending test was performed five times, and the material made of polyamide was broken when the bending test was performed 120 times, no fracture and breakage occurred in the product of this invention even when the product was bent 200 times.

In the repeated-bending test after water absorption, although the material made of polycarbonate was broken when the bending test was performed six times, and the material made of polyamide was broken when the bending test was performed 118 times, no fracture occurred in the product of example 1 even when the bending test was performed 250 times. In the resin material (molded article) of this invention, the bending strength was 150 MPa, the elongation was not less than 15 mm, and the water absorption rate was 0.05 to 0.2 wt %, and very good values were obtained. Furthermore, the experiment results for the workability, coloring, and the like were also good as indicated in Table 1.

The test for coloring will be described in more detail. The “coloring ΔE curry” in the second line from the bottom in Table 1 represents, as a numerical value, a result of a test in which a resin piece as a sample was immersed, in hot water in which curry powder was dissolved, at 37° C. for one week, the resin piece as the sample was then taken out, and the degree of coloring by the curry powder solution was examined by using a dedicated testing machine. The less the numerical value is, the less the degree of the coloring is, that is, the less discoloring is. Furthermore, “coloring ΔE fuchsin” represents a result of a similar test in which fuchsin was used as a colorant instead of the curry powder. The less the numerical value is in the test, the less discoloring is. As is apparent from the results, the value in the test with “coloring ΔE curry” was 1.29 in example 1, the value in the test with “coloring ΔE fuchsin” was 5 in example 1, and the values were sufficiently small as compared with the values in the tests for polyamide and polycarbonate, and discoloring is less likely to occur in example 1.

### Example 2

Powdery PET resin was prepared as thermoplastic polyester resin instead of the acrylic resin used in example 1. The PET resin was a commercially available product obtained by polycondensation of terephthalic acid or dimethyl terephthalate, and ethylene glycol. Instead of the PET resin, another commercially available thermoplastic polyester such as PBT resin and PEN resin may be used.

Polycaprolactone (PCL) obtained by synthesis of ε-caprolactone ([C6H1002] (“2-oxepanone” homopolymer), and the PET resin having a weight ratio of 15% with respect to the polycaprolactone (PCL) were copolymerized with each other to generate copolymerized polycaprolactone. Adjustment was performed in the obtained copolymerized polycaprolactone such that the weight ratio of ascorbic acid was 0.8%, the weight ratio of titanium oxide was 0.009%, the weight ratio of a red pigment (for example, one or more selected from organic and inorganic pigments such as aluminum lake and red iron oxide, which are certified colorants) was 0.003%, and the weight ratio of zinc stearate was 0.018%, with respect to the polycaprolactone (PCL).

Specifically, for use in a denture base gum part, the weight ratio of the PET resin was 20% (10 kg), the weight ratio of powdery silica was 15% (7.5 kg), the weight ratio of ascorbic acid was 0.8% (400 g), the weight ratio of titanium oxide was 0.009% (4.5 g), the weight ratio of the red pigment was 0.003% (1.5 g), and the weight ratio of zinc stearate was 0.018% (9 g), with respect to polycaprolactone (PCL) (50 kg).

The same evaluation test as in example 1 was performed for the above-described dental molded article (dental molded article substituting for gums or the like) of example 2. The results are indicated in Table 1. As indicated in Table 1, almost the same results as in example 1 were obtained for the dental molded article of example 2.

### DESCRIPTION OF THE REFERENCE CHARACTERS

1 partial denture
1a denture base
2 crown (dental prosthesis)
3 aesthetic laminate (dental prosthesis)
4 orthodontic appliance
5 mouthpiece (dental temporary crown)
6, 7 full dentures
6a denture base repairing material
6b denture base stabilizer

1. A dental molded article that has a predetermined shape, that is used in an oral cavity, and that contains polycaprolactone, the dental molded article comprising:
acrylic resin made of a polymer of acrylic acid ester, a polymer of methacrylic acid ester, or a copolymer of acrylic acid ester and methacrylic acid ester, the acrylic resin having a weight ratio of 5 to 30% with respect to the polycaprolactone;
powdery silica having a weight ratio of 5 to 25% with respect to the polycaprolactone;
ascorbic acid having a weight ratio of 0.3 to 1.8% with respect to the polycaprolactone;
titanium oxide having a weight ratio of 0.001 to 0.02% with respect to the polycaprolactone;
a pigment having a weight ratio of 0.001 to 0.1% with respect to the polycaprolactone;
zinc stearate having a weight ratio of 0.001 to 0.1% with respect to the polycaprolactone; and
a remainder being the polycaprolactone and unavoidable impurities.

2. (canceled)

3. A dental molded article that has a predetermined shape, that is used in an oral cavity, and that contains polycaprolactone, the dental molded article comprising:
thermoplastic polyester resin having a weight ratio of 5 to 30% with respect to the polycaprolactone;
powdery silica having a weight ratio of 5 to 25% with respect to the polycaprolactone;
ascorbic acid having a weight ratio of 0.3 to 1.8% with respect to the polycaprolactone;
titanium oxide having a weight ratio of 0.001 to 0.02% with respect to the polycaprolactone;
a pigment having a weight ratio of 0.001 to 0.1% with respect to the polycaprolactone;
zinc stearate having a weight ratio of 0.001 to 0.1% with respect to the polycaprolactone; and
a remainder being the polycaprolactone and unavoidable impurities.

4. (canceled)

5. A dental resin material that is used in accordance with a dental molded article used in an oral cavity, and that contains polycaprolactone, the dental resin material comprising:
acrylic resin made of a polymer of acrylic acid ester, a polymer of methacrylic acid ester, or a copolymer of acrylic acid ester and methacrylic acid ester, the acrylic resin having a weight ratio of 5 to 30% with respect to the polycaprolactone;
powdery silica having a weight ratio of 5 to 25% with respect to the polycaprolactone; and
a remainder being the polycaprolactone and unavoidable impurities.

6. (canceled)

7. A dental resin material that is used in accordance with a dental molded article used in an oral cavity, and that contains polycaprolactone, the dental resin material comprising:
thermoplastic polyester resin having a weight ratio of 5 to 30% with respect to the polycaprolactone;
powdery silica having a weight ratio of 5 to 25% with respect to the polycaprolactone; and
a remainder being the polycaprolactone and unavoidable impurities.

8. (canceled)

9. The dental molded article according to claim 1, wherein
the acrylic resin has a weight ratio of 10 to 25% with respect to the polycaprolactone, and
the powdery silica has a weight ratio of 5 to 20% with respect to the polycaprolactone.

10. The dental molded article according to claim 3, wherein
the thermoplastic polyester resin has a weight ratio of 10 to 25% with respect to the polycaprolactone, and
the powdery silica has a weight ratio of 5 to 20% with respect to the polycaprolactone.

11. The dental resin material according to claim 5, wherein
the acrylic resin has a weight ratio of 10 to 25% with respect to the polycaprolactone, and
the powdery silica has a weight ratio of 5 to 20% with respect to the polycaprolactone.

12. The dental resin material according to claim 7, wherein
the thermoplastic polyester resin has a weight ratio of 10 to 25% with respect to the polycaprolactone, and
the powdery silica has a weight ratio of 5 to 20% with respect to the polycaprolactone.
